(19) United States
(12) Patent Application Publication
(10) Pub. No.: US 2020/0077921 A1
(43) Pub. Date: Mar. 12, 2020

(54) Pen like device for detecting cancerous cells during surgery

(71) Applicant: Mohamed A. Basiony, Kenmore, WA (US)

(72) Inventor: Mohamed A. Basiony, Kenmore, WA (US)

(21) Appl. No.: 16/550,247

(22) Filed: Aug. 25, 2019

Related U.S. Application Data
(60) Provisional application No. 62/728,694, filed on Sep. 7, 2018.

(51) Int. Cl. A61B 5/053; A61B 5/05

(52) U.S. Cl. CPC A61B 5/0536; A61B 5/055; A61B 5/0522

(57) ABSTRACT

A pen like device for contactless imaging of the electrical conductivity of a target tissue during a surgery to different between cancerous cells and normal cells. The device operation is based on the principal of conductivity imaging by a magnetic induction. The device has a cylindrical transmitter coil and two receiver coils that are wound on a cylindrical rod of an electrically insulating material. The measured contactless conductivity through a non-electrically conductive wall of the device's body is displayed on a rotatable screen to help surgeons to make immediate decisions during a surgery.

### BACKGROUND

Surgeon needs to make immediate decisions during surgery as he/she can't wait a day or more that it takes for a routine processing and histology of a biopsy specimen. He or she will request an intra-operative (during surgery) pathology consult. This is often called a frozen section exam. After a frozen section exam is done, a fresh tissue specimen is sent from an operating room right to a pathologist.

As a patient is often under general anesthesia, it is important that a tissue specimen be looked at quickly. It usually takes 10 to 20 minutes as a fresh tissue specimen is grossly examined by a pathologist to decide which part of it should be looked at under a microscope. In this case instead of processing a tissue in wax blocks (normal procedure), a tissue is quickly frozen in a special solution that forms what looks like an ice cube around the tissue.

It is then sliced on a special machine, quickly dipped in a serious of dyes and looked under a microscope. Frozen sections usually do not show features of a tissue as clearly as sections of tissues embedded in wax, but they are a quickest way to help surgeons make decisions.

Thus, there is a requirement for a rapidly and simply method with less cost to detect cancerous cells directly during surgery.

### SUMMARY

It is an object of this implementation to provide a rapid, simple and easy technique for detecting a presence of cancerous cells during surgery using a pen like device.

It is another object of the implementation to provide a pen like device for detecting a presence of cancerous cells using a differential coil sensor to image a target tissue's conductivity by sensing magnetic fields of induced eddy currents.

It is still another object of this implementation to provide a pen like device for detecting a presence of cancerous cells by a magnetic excitation technique to induce eddy current inside a target tissue and resulting magnetic fields are measured with receiver (pick-up) coils.

It is still another object of this implementation to provide a diagnostic tool for a surgeon to evaluate if all cancerous cells have been removed during surgery.

So, the present implementation is based on a conductivity imaging by a magnetic induction. Imaging a conductivity by a magnetic induction has been widely used as the rationale is that it is a contactless measurement, as a device measures a conductivity contactlessly through its a non-electrically conductive wall.

A pen like device according to the present implementation comprises a transmitter coil for applying an excitation current to a target tissue and two receiver coils for receiving an induced current generated by the excitation current via a target tissue for providing an output signal for computing the conductivity of a target tissue. A transmitter coil and two receiver coils are embodied as cylindrical coils, are wound on a cylindrical rod of an electrically insulating material and are extended on a cylindrical lateral surface of a cylindrical rod in such a manner that the cylinder axes of a transmitter coil and two receiver coils coincide with one another and with a rod axis. Two receiving coils are arranged axially one after the other and at a same distance from a transmitter coil on oppositely lying sides of a transmitter coil.

Also, a pen like device comprises a device body with electronics inside, an on/off switch, an alarm reset button, a rotatable screen and a power cable.

### BRIEF DESCRIPTION OF THE DRAWINGS

The accompanying drawings, which are incorporated in, and constitute a part of the specification, illustrate or exemplify embodiment of the present implementation and, together with the description, generally explain the principles and features of the present implementation. The drawings are briefly described as follows:

FIG. 1 illustrates a schematic representation of a differential coil sensor.

FIG. 2 Illustrates a perspective view of a pen like device during a surgical operation.

FIG. 3 illustrates a perspective view of components of the device of FIG. 2.

### DETAILED DESCRIPTION

The following detailed description illustrates the principal of the disclosure by way of example not by way of limitation. While a reference use of the present disclosure describes a pen like device to be used for detecting a presence of cancerous cells during a surgery, additional non-limiting usage would also include a conductivity imaging via contactless measurements, as those of ordinary skill in the art will readily understand.

The present implementation is directed to a pen like device for rapidly distinguishing cancerous cells from normal cells using a contactless conductivity imaging technique.

The present implementation is based on conductivity imaging using a differential coil sensor to image a target tissue's conductivity by a magnetic induction. In this method a magnetic excitation is used to induced eddy currents inside a target tissue and the resulting magnetic fields are measured with receiver coils.

The following detailed description illustrates the principal:

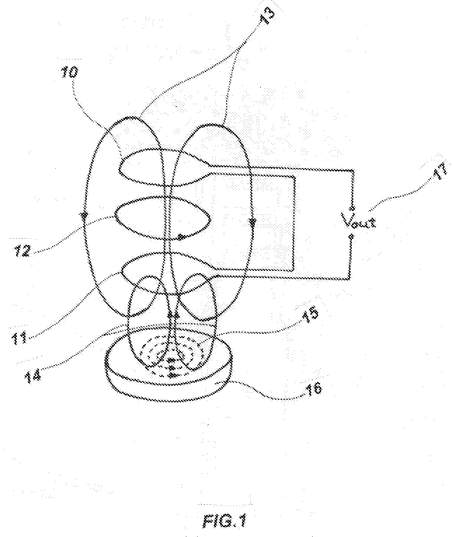

FIG. 1 illustrates a differential coil sensor to image a tissue's conductivity by sensing magnetic fields of induced eddy current, wherein a transmitter coil 12 generates a primary flux 13 that induces eddy currents 15 in a target tissue 16. Primary flux 13 that is linked through two identical receiver coils 10 and 11, is equal since both receiver coils 10 and 11 are at a same distance from a transmitter coil 12 which is the source of a primary flux 13.

However, a secondary flux 14 that is generated by eddy currents 15 flowing in a target tissue 16 is linked through differentially connected receiver coils 10 and 11 unequally or in an unbalanced fashion due to the fact that a flux strength is diminished with distance. This caused a gradient voltage 17 in receiver coils 10 and 11 due to a conductive target tissue 16. This gradient voltage 17 is directly proportional to a conductivity of a target tissue 16 and it may act as measure for a conductivity of a target tissue 16.

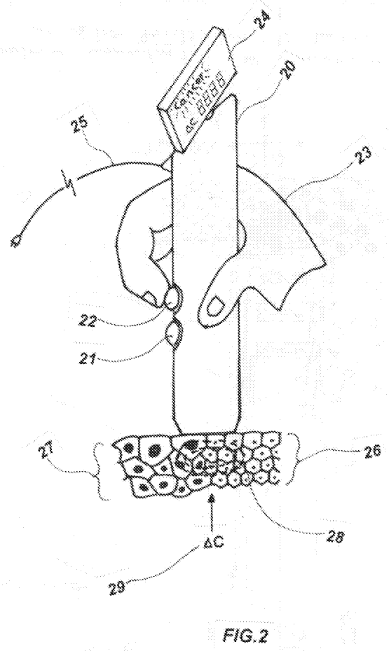

FIG. 2 illustrates a pen like device during a surgery wherein a device body 20 with on/off switch 22 to power a device and an alarm/reset button 21. An alarm/reset button is used to reset an audible alarm in case a device detects cancerous cells. Also, as can be seen in FIG. 2, a user hand 23, a rotatable screen 24 and a power cord 25.

A rotatable screen 24 is rotatable to allow for a user to easily see a displayed information. A displayed information is “cancer” or “normal” cells plus a differential conductivity imaging ΔC 29, which is a differential conductivity between normal cells 26 and cancerous cells 27. The displayed information either “cancer” or “normal” based on a data stored in a device's memory (not shown for simplicity) that compares a measured conductivity with a stored data to differentiate between “cancer” and “normal” cells, while a differential conductivity imaging ΔC 29, is based on change in a conductivity reading between cancerous cells 27 and normal cells 26.

Those of ordinary skill in this art will readily can see that cancerous cells 27 have large number of irregularly shaped dividing cells, large and variable shaped nuclei, variation in cell size and shape, small cytoplasmic volume relative to nuclei, loss of normal specialized cell features, disorganized arrangement of cells and poorly defined tumor boundary. All these differences between cancerous cells 27 and normal cells 26 may be reflected into a differential conductivity imaging ΔC 29 and into “cancer” cells detection quickly during surgery.

Also, FIG. 2 illustrates eddy currents 28 induced by a primary flux 13 of a transmitter coil 12 inside a device (not shown for simplicity).

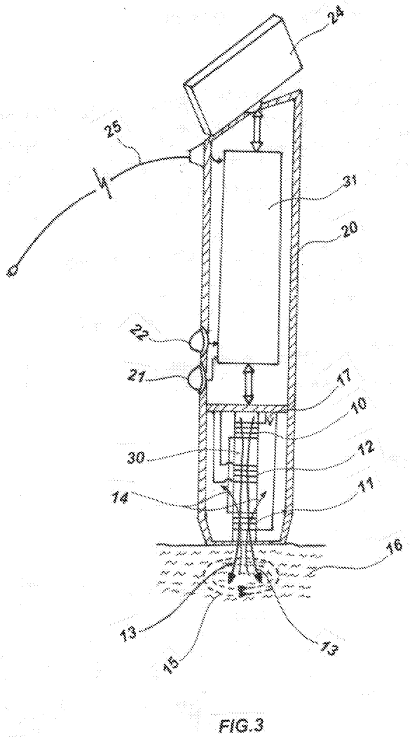

FIG. 3 illustrates device's components, wherein a device body 20, an on/off switch 22, an alarm/rest button 21, a rotatable screen 24 and a power cord 25. A device also has a transmitter coil 12 and two receiver coils 10 and 11. A transmitter coil 12 and two receiver coils 10 and 11 are formed by a conductive material like aluminum, copper, silver or the like and are wound on a cylindrical rod 30. A cylindrical rod 30 and a device body 20 are made of electrically non-conductive material like plastic. The windings of a transmitter coil 12 and two receiver coils 10 and 11 are extended on a cylindrical lateral surface of a cylindrical rod 30. Also, a transmitter coil 12 and two receiver coils 10 and 11 are arranged axially one after the other and two receiver coils 10 and 11 are at the same distance from a transmitter coil 12 on oppositely lying sides of a transmitter coil 12.

After a device's activation, a transmitter coil 12 receives an alternating current via a power cord 25 to generate a primary flux 13 to produce eddy currents 15 in a target tissue 16 through a non-electrically conductive wall of a device body 20. Secondary flux 14 generated by eddy currents 15 flowing in a target tissue 16 is linked through differentially connected receiver coils 10 and 11.

This caused a gradient voltage 17 which is directly proportional to a conductivity of a target tissue 16. The gradient voltage 17 is proceeded to an electronic circuit 31 to be displayed as a differential conductivity imaging ΔC 29 on a rotatable screen 24 and also as a “cancer” or “normal” based on a conductivity imaging of normal cells 26 and cancerous cells 27.

In case a device detects “cancer” cells, it will give an audible alarm that is reset bay the user by pressing an alarm/rest button 21. So, this imaging of a target tissue's conductivity may help surgeons to make immediate decisions during surgery.

1. A pen like device comprising:
a differential coil sensor comprising a transmitter coil for applying an excitation current to a target tissue and two receiver coils for receiving an induced current generated by the excitation current via a target tissue for providing an output signal for computing the conductivity of a target tissue. Said transmitter coil and said two receiver coils are embodied as cylindrical coils, are wound on a cylindrical rod of an electrically insulating material and are extended on a cylindrical lateral surface of said cylindrical rod in such a manner that cylinder axes of said transmitter coil and said two receiver coils coincide with one another and with a cylindrical rod axis. Said two receiving coils are arranged axially one after the other and at the same distance from said transmitter coil on oppositely lying sides of said transmitter coil;
a device body, a rotatable screen, an on/off switch, an alarm/reset button, a power cord and an electronic circuit.

2. The device of claim 1, wherein said device operation based on a contactless conductivity imaging by a magnetic induction technique.

3. The device of claim 1, wherein said transmitter coil and said two receiver coils are formed by a conductive material.

4. The device of claim 1, wherein said rotatable screen is rotatable.

5. The device of claim 1, wherein a displayed information is “cancer” or “normal” and a differential conductivity imaging of cancerous cells and normal cells.
